(19) United States
(12) Patent Application Publication
(10) Pub. No.: US 2023/0042710 A1
(43) Pub. Date: Feb. 9, 2023

(54) ELECTROCHEMICAL PROXIMITY ASSAY

(71) Applicants: INNAMED, INC., Philadelphia, PA (US); AUBURN UNIVERSITY, Auburn, AL (US)

(72) Inventors: Katarena I. FORD, Auburn, AL (US); Niamat E. KHUDA, Auburn, AL (US); Asanka GURUKANDURE GEDARA, Auburn, AL (US); Christopher J. EASLEY, Auburn, AL (US); Subramaniam SOMASUNDARAM, Auburn, AL (US); Anup SINGH, Philadelphia, PA (US); Eshwar INAPURI, Philadelphia, PA (US)

(73) Assignees: INNAMED, INC., Philadelphia, PA (US); AUBURN UNIVERSITY, Auburn, AL (US)

(21) Appl. No.: 17/789,161

(22) PCT Filed: Dec. 31, 2020
(86) PCT No.: PCT/US2020/067760

§ 371 Date: Jun. 24, 2022

Related U.S. Application Data
(60) Provisional application No. 62/957,099, filed on Jan. 3, 2020.

(51) Int. Cl. C12Q 1/6825; G01N 27/327

(52) U.S. Cl. CPC C12Q 1/6825; G01N 27/3276; G01N 27/3277

(57) ABSTRACT

Described herein are nucleic acid-based electrochemical proximity assays (ECPAs) for sample quantification. The invention may also include a biosensor with a sensing mechanism that uses a pair of aptamers or antibodies that bind the target of interest. More specifically, the invention relates to an electrochemical-based read out of a sensing mechanism that uses a nucleic acid-based proximity assay in conjunction with a pair of aptamers or antibodies for sample quantification. The biosensor or a set of biosensors can be used either as a standalone measurement system for a single analyte target or as a component of a multiplexed cartridge for multiple analytes.

### CROSS REFERENCE TO RELATED APPLICATIONS

This patent application claims priority to U.S. Provisional Patent Application No. 62/957,099, titled “ELECTROCHEMICAL PROXIMITY ASSAY” and filed on Jan. 3, 2020, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### STATEMENT AS TO FEDERALLY SPONSORED RESEARCH

This invention was made with Government support under Grant No. R01 DK093810 awarded by the National Institutes of Health, Grant No. CBET-1403495 awarded by the National Science Foundation and Contract #HHSO100201800040C by Biomedical Advanced Research and Development Authority. The Government has certain rights in the invention.

### FIELD

Described herein are assays capable of quantifying peptide and protein biomarkers and small molecules in a test sample. More specifically, described herein are electrochemical proximity assay-based biosensors that may include a sensing mechanism that uses a complex of nucleic acid-based and biologic reagents for sample quantification. The biosensor can be used either as a standalone measurement system or as a component of a testing cartridge that measures fluid analyte concentrations in a multiplexed manner.

### BACKGROUND

Diagnostics is a critical component of patient care. Among the types of biological targets measured by traditional diagnostics, sensitive detection of proteins is important due to their clinical relevance in diagnosing the onset or progression of particular disease states. Unless specialized point-of-care assays are available for the protein of interest, quantitation is typically performed in a centralized laboratory by technicians which is slow and expensive.

Despite the promise of existing point-of-care assay technologies for protein detection, implementing traditional sandwich ELISA assays is difficult due to the heterogeneous format of the assay. Nonetheless, the flexibility of the dual-antibody recognition concept is highly valuable to sensitive and specific detection. Other methods such as proximity immunoassays (e.g. proximity ligation assay) or the molecular pincer assay can overcome some of the limitations of ELISA with their homogeneous assay format and sensitive detection limits. Nonetheless, limitations in current proximity assays impede their use in a point-of-care setting due to the required qPCR for signal amplification to enable sensitive detection and the associated relatively high cost of these assays.

For measurement, electrochemical detection is well-suited for point-of-care devices (e.g. iSTAT, glucometer) as it offers great signal stability, simple instrumentation, high sensitivity, ease of calibration and compatibility with miniaturization.

The present disclosure builds upon the existing electrochemical proximity assay (ECPA), a simple, highly sensitive method for protein quantification. U.S. Pat. No. 9,335,292 illustrates one example of ECPA. Such prior iterations of ECPA suffer from issues such as high background and low signal, slow hybridization kinetics and suboptimal environmental and electrical testing parameters. It would be helpful to provide electrochemical proximity assays that address these problems and that are optimized for use in a commercial, point-of-care diagnostic system.

### SUMMARY OF THE DISCLOSURE

Described herein are electrochemical proximity assays (ECPA), including methods and systems, that may provide higher signal, lower background and more reliable results than traditional ECPA assays. In general, these assays are nucleic acid-based electrochemical proximity assays for quantification of target analytes (including, but not limited to, proteins, polynucleotides, such as mRNA, microRNA, DNA, etc.). These electrochemical proximity assays may be performed on the surface of an electrically conductive base, such as a gold electrode or other substrate, to form a biosensor.

The electrochemical proximity assays described herein outline methods and systems in which the capture probe may be pre-incubated with a thiol polynucleotide (e.g., DNA) immobilized to the electrode surface to form a nucleic acid layer, and separating pre-incubating a redox-conjugated DNA and detection probe to form an ECPA probe, then combining the ECPA probe, nucleic acid layer and a sample including the target in order to detect an electrochemical signal in the electrode when the ECPA probe forms a complex with the target and the nucleic acid layer. In any of these ECPA assays described herein, the gap between the redox molecule(s) of the ECPA probe and the conductive base forms a gap that is optimized by salt concentration for maximum signal (current), e.g., to be between 3-8 nucleotides (e.g., between 3-5, about 4, etc.).

In some variations, these electrochemical proximity assays may include involves the formation of a multi-part (e.g., four-part, five-part, etc.) complex that may include, e.g., a polynucleotide tethered to the conductive base (e.g., a thiol DNA), a capture probe (e.g., antibody, aptamer, etc.), a target analyte, a detection probe (e.g., antibody, aptamer, etc.) and an polynucleotide linked to a redox molecule (e.g., MB-DNA) for signal and electron transfer with the electrode. The electrochemical proximity assays described herein have been optimized to minimize the background and maximize the signal through modifications in the assay workflow and hybridization conditions. These assays may combine readout from several such biosensors each using a different set of binding probes and conditions for measurement.

In particular, described herein are assays in which the distance between the one or more redox molecules of the ECPA probe and the electrically conductive base is separated by between about 3 and 5 nucleotides when the ECPA probe is hybridized to a complementary region of the nucleic acid layer extending from the electrically conductive base. The separation distance between the redox molecule(s) and the electrically conductive base when detecting a target molecule may be optimized to the salt concentration present.

Also described herein are ECPA assays that are optimized by the order in which the various components forming the hybridized ECPA complex are combined.

In general, an ECPA assay is a separation-free, electrochemical assay that may include a direct readout that is amenable to highly sensitive and selective quantitation of a wide variety of target proteins. The first generation of the electrochemical proximity assay (ECPA) included to target-binding molecules (e.g., thrombin aptamers) which formed a cooperative complex only in the presence of target molecules, bringing a redox molecule (e.g., methylene blue, or MB) that was conjugated to oligonucleotide close to a gold electrode, typically not separated from the conductive surface by any appreciable distance (e.g., less than 0.5 nm). Without washing steps, electrical current was increased in proportion to the concentration of a specific target protein. The addition of a short DNA competitor was used to reduce background current of the MB peak to a baseline levels. Typically, a single redox molecule was included.

As used herein, the terms “antibody” and “antibodies” can include, but are not limited to, monoclonal antibodies, polyclonal/multispecific antibodies, human antibodies, humanized antibodies, camelised antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (e.g., anti-Id antibodies to antibodies of the disclosure), and epitope-binding fragments of any of the above. In particular, antibodies include immunoglobulin molecules and immunologically active fragments of immunoglobulin molecules (e.g., molecules that contain an antigen binding site). Immunoglobulin molecules can be of any type (e.g., IgG, IgE, IgM, IgD, IgA and IgY), class (e.g., IgG1, IgG2, IgG3, IgG4, IgA1 and IgA2), or subclass. The antibodies may be from any animal origin including birds and mammals (e.g., human, murine, donkey, sheep, rabbit, goat, guinea pig, camel, horse, or chicken). Preferably, the antibodies are human or humanized monoclonal antibodies. As used herein, “human” antibodies include antibodies having the amino acid sequence of a human immunoglobulin and include antibodies isolated from human immunoglobulin libraries or from mice that express antibodies from human genes. The antibodies may be monospecific, bispecific, trispecific, or of greater multispecificity.

As used herein, aptamers may be high affinity, high specificity polypeptide, RNA, or DNA-based probes produced by in vitro selection experiments. Aptamers may be generated from random sequences of nucleotides or amino acids, selectively screened by absorption to molecular antigens or cells, and enriched to purify specific high affinity binding ligands, for example. In solution, aptamers may be unstructured but may fold and enwrap target epitopes providing specific binding recognition. The unique folding of the nucleic acids around the epitope, for example, affords discriminatory intermolecular contacts through hydrogen bonding, electrostatic interaction, stacking, and shape complementarity.

Aptamers must also be differentiated from the naturally occurring nucleic acid sequences that bind to certain proteins. These latter sequences generally are naturally occurring sequences embedded within the genome of the organism that bind to a specialized sub-group of proteins or polypeptides, or their derivatives, that are involved in the transcription, translation, and transportation of naturally occurring nucleic acids, i.e., protein-binding nucleic acids.

Aptamers on the other hand are short, isolated, non-naturally occurring nucleic acid molecules. While aptamers can be identified that bind nucleic acid-binding proteins, in most cases such aptamers have little or no sequence identity to the sequences recognized by the nucleic acid-binding proteins in nature. More importantly, aptamers can be selected to bind virtually any protein (not just nucleic acid-binding proteins) as well as almost any target of interest including small molecules, carbohydrates, peptides, etc. For most targets, even proteins, a naturally occurring nucleic acid sequence to which it binds may not exist. For those targets that do have such a sequence, i.e., nucleic acid-binding proteins, such sequences may differ from aptamers as a result of the relatively low binding affinity used in nature as compared to tightly binding aptamers. Aptamers are capable of specifically binding to selected targets and modulating the target's activity or binding interactions, e.g., through binding, aptamers may block their target's ability to function. The functional property of specific binding to a target is an inherent property of an aptamer. Proximity assays, such as the method described herein, are capable of functioning with any molecular recognition elements, which includes aptamers, nucleic acid binding proteins, antibodies, etc.

As used herein, a “nucleotide” refers to an organic molecule consisting of a nucleotide and a phosphate. They serve as monomeric units of the nucleic acid polymers deoxyribonucleic acid (DNA) and ribonucleic acid (RNA). Nucleotides are composed of three subunit molecules: a nucleobase, a five-carbon sugar (ribose or deoxyribose), and a phosphate group consisting of one to three phosphates. The four nucleobases in DNA are guanine, adenine, cytosine and thymine; in RNA, uracil is used in place of thymine. On average, each (single stranded) nucleotide may have a length of about 0.6 nm.

A “polynucleotide” or “nucleic acid” generally refer to a string of at least two base-sugar-phosphate combinations. As used herein, the terms include deoxyribonucleic acid (DNA) and ribonucleic acid (RNA) and generally refer to any polyribonucleotide or polydeoxyribonucleotide, which may be unmodified RNA or DNA or modified RNA or DNA. RNA may be in the form of a tRNA (transfer RNA), snRNA (small nuclear RNA), rRNA (ribosomal RNA), mRNA (messenger RNA), anti-sense RNA, RNAi (RNA interference construct), siRNA (short interfering RNA), or ribozymes. Thus, for instance, polynucleotides as used herein refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. The terms “nucleic acid sequence” and “oligonucleotide” also encompasses a nucleic acid and polynucleotide as defined above. The term “DNA molecule” includes nucleic acids/polynucleotides that are made of DNA.

In addition, polynucleotide as used herein refers to single-stranded or double-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term polynucleotide as it is employed herein embraces such chemically, enzymatically or metabolically modified forms of polynucleotides, as well as the chemical forms of DNA and RNA characteristic of viruses and cells, including simple and complex cells, inter alia. For instance, the term polynucleotide includes DNAs or RNAs as described above that contain one or more modified bases. Thus, DNAs or RNAs comprising unusual bases, such as inosine, or modified bases, such as tritylated bases, to name just two examples, are polynucleotides as the term is used herein.

The term may also include PNAs (peptide nucleic acids), phosphorothioates, and other variants of the phosphate backbone of native nucleic acids. Natural nucleic acids have a phosphate backbone, artificial nucleic acids may contain other types of backbones, but contain the same bases. Thus, DNAs or RNAs with backbones modified for stability or for other reasons are “nucleic acids” or “polynucleotides” as that term is intended herein.

Described herein are electrochemical assays that provide highly sensitive and selective quantitation of a variety of targets (e.g., target proteins), and in particular, include electrochemical proximity assays (ECPAs) which may leverage two specific binding agents (e.g., two aptamers, two antibody-oligonucleotide probes, combinations of these, etc., including any other specific binding agent), and proximity-dependent DNA hybridization to move one (or more preferably more, e.g., 2, 3, 4, 5, 6, 7, 8, etc.) redox active molecule(s) to within a predefined distance of an electrically conductive base. The ECPAs described herein may produce a rapid, quantitative result (e.g., within a few minutes, such as five minutes or less), enabling point-of-care use in the detection of biomarkers of disease.

The ECPAs described herein may include exposing a mixture of an ECPA probe and a target to a conductive base onto which nucleic acid layer has been formed. The ECPA probe may comprise a polynucleotide coupled to a redox molecule. The ECPA may also include generating an electrical (also referred to as electrochemical) signal in the conductive base by forming a complex of the nucleic acid layer, the ECPA probe, and the target and binding the polynucleotide of the ECPA probe to a complementary polynucleotide of the nucleic acid layer on the conductive base, so that the redox molecule of the ECPA probe is separated from the conductive base by a predefined and optimized distance, e.g., between 3 and 8 (e.g., between 3 and 5, etc.) nucleotides of the complementary polynucleotide of the nucleic acid layer. Thereafter, the amount of the target may be quantified by analyzing the electrochemical signal, wherein the electrochemical signal changes in proportion to changes in the concentration of the target.

In some variations, the method may include forming the nucleic acid layer on the electrically conductive base (substrate). The electrochemical signal may be generated by immersing the electrically conductive base comprising the nucleic acid layer into a solution comprising the ECPA probe and target. The nucleic acid layer, ECPA probe, and target may form a complex. The amount of the target may be quantified by analyzing the electrochemical signal; the electrochemical signal typically changes (e.g., increases or decreases) in proportion to changes in the concentration of the target.

The nucleic acid layer may comprise at least one surface immobilized nucleic acid strand (e.g., surface immobilized DNA strand). The surface immobilized nucleic acid may be a surface immobilized DNA, such as a thiolated DNA, an amine labeled DNA, an RNA, a modified RNA, and a combination thereof.

An ECPA in which the surface immobilized DNA comprises thiolated DNA may form a self-assembled monolayer (SAM) on an electrically conductive base (e.g., a gold electrode). The self-assembly of thiolated DNA strands onto the electrically conductive base may be accomplished via an alkanethiol moiety at the 5′ terminus. In some variations, an ECPA may include a nucleic acid layer that is formed by covalent attachment of the nucleic acid to the electrically conductive base. The electrically conductive base (substrate) may be selected from, but not limited to, a metal electrode (e.g., gold, platinum), an activated carbon electrode, a conductive ceramic, a conductive glass, and any combination thereof.

An ECPA probe may include a molecular recognition element specific to the target (e.g., a specific binding agent, such as an antibody or portion of an antibody, aptamer, etc.) and at least one nucleic acid/electron transfer conjugate (e.g., equivalently referred to as a redox molecule, redox active molecule, or redox agent); the electron transfer element (e.g., oxidized or reduced) may be any electrochemically active molecule, for example, but not limited to, methylene blue (MB), ferrocene/ferricinium, tris(2-2′-bipyridine)Ru(II), quinone/hydroquinone, and their derivatives, and any combination thereof. As mentioned above, it may be preferable to use multiple such redox agents, such as 2, 3, 4, 5, 6, 7, 8, 9, 10, 11, 12, 13, 14, 15, etc. The optimal number may be, e.g., between 3-15; the endogeneous concentration of the target of interest and the sensitivity of the probes is to be considered for optimal number of redox molecules. For example, in some variations, five (5) redox molecules are effective at increasing the signal without unduly increasing the background (e.g., false positive) signal, particularly when use as described herein, and as compared to a single redox molecule. The one or preferably more redox molecules may be coupled to the ECPA probe so that they are presented at a specific distance from the conductive base when the ECPA probe is bound to the nucleic acid layer.

As mentioned, the ECPA may include a molecular recognition element that is selected from an aptamer, an antibody, an antibody/DNA conjugate, and/or a combination thereof.

Although the ECPA methods and systems (including kits) described herein may be used with a competitor (e.g., a short, single stranded nucleic acid competitor, such as a short single stranded DNA competitor), such competitors have been found to be unnecessary, particularly in light of the improvements described herein. Prior versions of ECPA used a nucleic acid competitor having complimentary bases with the surface immobilized nucleic acid (e.g., 5 to 50 complimentary bases with the surface immobilized nucleic acid).

Generally, the improved ECPA methods and systems described herein provide robust assays, which have a lower background and a higher specific signal as compared to known assays, including previously described ECPA methods and systems. Specifically, the surprising benefit of optimizing the distance (spacing) between the redox agent, and in particular, multiple redox agents, has been found to decrease the background by more than eight-fold (e.g., between 8 times and 13 times) compared to what would be expected from previously described ECPA assays. The combination of the optimal spacing (e.g., between about 3 and 8 nucleotides of the first polynucleotide of the nucleic acid layer, which may be e.g., between about 1.1 and 3 nm), in some examples in a buffer solution having a salt concentration of between 0.05 to 0.75 M may result in significantly greater signal to noise ratios over a much larger sensitivity range. Further, the improved ECPA methods and systems described herein may have a much larger linear concentration range detected, and a larger dynamic range; for comparison the ECPA methods and systems described herein may have a dynamic range for detection of target that is more than three orders of magnitude greater than the dynamic range seen without the improvements described herein. This dependency of the dynamic range on the spacing and number of the redox agents (and/or the salt concentration) is both surprising and consistent across a variety of different targets.

Any of the improved ECPA methods and systems described herein may include about 5 redox molecules (e.g., methylene blue) that are conjugated to an ECPA probe, e.g., to a polynucleotide of the ECPA probe, so that the redox molecules are about 4 nucleotides (e.g., about 1.1-1.6 nm) of the first polynucleotide of the nucleic acid layer from where it is attached to the conductive base (e.g., substrate).

For example, described herein are electrochemical proximity assay (ECPA) systems that include: a nucleic acid layer comprising a capture probe and a first polynucleotide conjugated to a conductive base, wherein the capture probe comprises: a molecular recognition element configured to specifically bind to a target, and a second polynucleotide having a first region that is complementary to a second region of the first polynucleotide; and an ECPA probe comprising a redox-conjugated polynucleotide conjugated to a detection probe, wherein the redox-conjugated polynucleotide comprises a plurality of redox molecules conjugated to a third polynucleotide, and wherein the detection probe comprises a molecular recognition element configured to specifically bind to the target and coupled to a fourth polynucleotide having a third region that is complementary to a fourth region of the redox-conjugated polynucleotide, wherein the ECPA probe and the nucleic acid layer are configured to form a complex with the target wherein the redox molecule of the ECPA probe is separated from the conductive base by between 3 to 8 nucleotides of the first polynucleotide of the nucleic acid layer.

For example, an electrochemical proximity assay (ECPA) system may include: an assay chamber comprising a nucleic acid layer comprising a capture probe and a first polynucleotide conjugated to a conductive base of the chamber, wherein the capture probe comprises: a molecular recognition element configured to specifically bind to a target, and a second polynucleotide having a first region that is complementary to a second region of the first polynucleotide; and a first solution comprising an ECPA probe comprising a redox-conjugated polynucleotide conjugated to a detection probe, wherein the redox-conjugated polynucleotide comprises a plurality of redox molecules conjugated to a third polynucleotide, and wherein the detection probe comprises a molecular recognition element configured to specifically bind to the target and coupled to a fourth polynucleotide having a third region that is complementary to a fourth region of the redox-conjugated polynucleotide, wherein the ECPA probe and the nucleic acid layer are configured to form a complex with the target wherein the plurality of redox molecules of the ECPA probe are separated from the conductive base by between 3 to 5 nucleotides of the first polynucleotide of the nucleic acid layer.

The plurality of redox molecules may include methylene blue or any other appropriate redox molecule.

In any of these methods and systems, the plurality of redox molecules of the ECPA probe may be separated from the conductive base by a spacer formed by the portion of the first polynucleotide of the nucleic acid layer (the portion that is bound to the conductive base). This spacer may be, e.g., a stretch of adenosine nucleotides (e.g., a 3-8 polyA region). For example, the plurality of redox molecules of the ECPA probe may be separated from the conductive base by between 3 to 5 nucleotides of the first polynucleotide of the nucleic acid layer when the ECPA probe and the nucleic acid layer form a complex with the target. The 3-5 nucleosides may be referred to as a spacer, as it spaces the plurality of redox molecules at the end of the ECPA probe from the conductive layer when the ECPA probe is hybridized to the nucleic acid layer.

In any of these systems, the system may further include a buffer solution having a salt concentration of between 0.25 to 0.75 M. In some examples the redox molecule of the ECPA probe may be separated from the conductive base by between 5 to 8 nucleotides of the nucleic acid layer and the buffer may have a salt concentration between about 0.05 to 0.25 M.

In any of these systems the nucleic acid layer may include thiolated-DNA. The ECPA probe may include between 3 and 15 redox (e.g., five) molecules, such as 5 methylene blue molecules.

Any appropriate molecular recognition element may be used. For example, the capture probe may comprise an aptamer or an antibody. In general, although virtually any target may be used, including protein or peptide targets, specific and non-limiting examples of ECPA systems described herein include serum amyloid A-1 (SAA-1), MR-proADM, and NT-proBNP.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A schematically illustrates the ECPA assay described herein.

FIG. 1B is a schematic illustration of a series of poly(A) modified thiolated-DNA (e.g. polynucleotides) forming a part of a nucleic acid layer on a conductive base in which spacers of various lengths (expressed as 0-19 polynucleotides, e.g., 0A-19A) are shown.

FIGS. 2A-2D are graphs illustrating peak current versus the square wave voltammetry (SWV) frequency relationship for the different gap (spacing) distances, 0A-3A (FIG. 2A), 4-6A (FIG. 2B), 7-9A (FIG. 2C) and 11-14A (FIG. 2D).

FIG. 3 illustrates the relationship between the electrochemical critical time and the length of the spacing between the redox molecule(s) and the substrate when the ECPA complex is formed during the assay.

FIGS. 4A-4D illustrates one theoretical model for the apparent hindrance introduced by an electrical double layer on the surface during DNA hybridization in ECPA as described herein.

FIG. 5 is a graph showing the effect of salt concentration on optimal spacer lengths (e.g., spacing between the redox molecule(s) and the substrate during ECPA).

FIG. 6 illustrates the results of an optimization experiment for one example of ECPA comparing four possible backgrounds with the ECPA signal as described herein.

FIG. 7 is a graph illustrating the improvement in signal (above background) when increasing the number of redox molecules per ECPA probe in an exemplary ECPA assay as described herein.

FIGS. 8A-8B illustrates a comparison of the use of one redox molecule (e.g., one methylene blue, or 1 MB) vs. multiple, e.g., 5 redox molecules (e.g., five methylene blue, or 5 MB) in an ECPA assay for a target DNA molecule. FIGS. 8C and 8D illustrate respective signals, using square wave voltammetric measurements when detecting target DNA for ECPA with 1 MB and 5 MB, respectively. The signals shown in FIGS. 8C-8D are subtracted from the baseline.

FIGS. 9A-9B illustrates a comparison of one redox molecule (e.g., one methylene blue, or 1 MB) vs. multiple, e.g., 5 redox molecules (5 MB) in an ECPA assay for Serum Amyloid A-1, an acute-phase inflammatory protein. FIGS. 9C and 9D illustrate respective signals when detecting target Serum Amyloid A-1 for 1 MB and 5 MB, respectively. The signals shown in FIGS. 8C-8D are subtracted from the background.

FIG. 10 illustrates a generic model of an ECPA assay as described herein, using multiple (e.g., five) redox molecules.

FIG. 11 schematically illustrates one method of performing an ECPA assay as described herein.

FIG. 12A shows one example of a calibration curve for the midregional pro-adrenomedullin (MR-proADM) target using an ECPA assay as described herein with multiple (e.g., 5) redox molecules.

FIG. 12B illustrates the use of an assay as illustrated in FIG. 12A to detect MR pro-ADM concentration (in nM) from sepsis patients serum samples and serum samples from healthy patient samples.

FIG. 13A schematically illustrates one example of an ECPA complex for binding and detecting N-terminal (NT) pro hormone B-type natriuretic peptide (NT-proBNP) as described herein.

FIG. 13B is a chart illustrating the detection of NT-proBNP using the complex shown in FIG. 13A (in the presence of no NT-proBNP and 10 nM NT proBNP).

FIG. 14A illustrates an ECPA complex configured for detecting Serum Amyloid A-1 (SAA-1), similar to that shown in FIG. 9B.

FIG. 14B is a graph illustrating a calibration curve for quantifying SAA-1 using the ECPA complex shown in FIG. 14A in buffer. FIG. 14C is a graph showing the concentration of SAA-1 estimated using an ECPA complex such as that shown in FIG. 14A (applying the calibration curve of FIG. 14B) in plasma for known concentrations of SAA-1 in the plasma.

### DETAILED DESCRIPTION

The ECPA methods and assays (e.g., kits, systems, etc.) described herein may be used for identifying, detecting, and/or quantifying a target in a sample where the target is selected from a protein, a small molecule, a multi-protein complex, a nucleic acid, a polymer, a whole cell, a virus, a biological polymer, and a combination thereof. The target may cause the nucleic acid/electron transfer conjugate to be closer to a surface of the electrically conductive base and allow an electron transfer process. The complex may be re-usable, e.g., the complex may be used for measurement, then washed with a solvent and reused.

The ECPA methods and systems described herein may be use for the detection and/or treatment of health related issues including, but not limited to, heart attack, stroke, rhabdomylosis, fertility, diabetes, obesity, metabolic syndrome, sepsis, inflammatory response, food safety, tuberculosis, and any combination thereof. For example, described herein are methods for ECPA that may be used to detect and/or treat any disease and/or condition diagnosed by a protein or peptide, including rapidly detecting, identifying, and/or quantifying a target in a sample.

These methods may include forming the nucleic acid layer by immobilizing a first nucleic acid (polynucleotide) on an electrically conductive base and pre-incubating a capture probe to form a nucleic acid layer. The nucleic acid layer, including the bound capture probe (which may be bound by allowing complementary polynucleotides to hybridize, and may include, e.g., an aptamer or antibody/portion of an antibody that specifically binds to the target or a nucleic acid that binds to the target) may be formed by one or more pre-incubating steps prior to combining with the ECPA probe. Separately, the ECPA probe may be formed by mixing the redox-conjugated polynucleotide and the detection probe (e.g., an aptamer or antibody/portion of an antibody that specifically binds to the target). The ECPA probe and nucleic acid layer may later be combined with a solution containing (or suspected to contain) the target, e.g., by immersing the electrically conductive base of the nucleic acid layer with a solution comprising the ECPA probe and the sample solution (e.g., target solution) to generate an electrochemical signal that may be detected to identify, and/or quantify the target by analyzing the electrochemical signal. The electrochemical signal may change (e.g., increase or decrease) in proportion to the concentration of the target, even in the presence of complex backgrounds such as blood or urine.

In some examples, the systems and methods described herein may include a first solution including a capture probe (e.g., a molecular recognition element configured to specifically bind to a target, and a second polynucleotide) that is hybridized to a thiolated first polynucleotide. This first solution is the proto nucleic acid layer that will be combined with the electrode to form the nucleic acid layer. The first (proto nucleic acid layer) solution may include a buffer (e.g., a HEPES buffer). The system may also include a second solution including the ECPA probe, which may be formed of a mixture of the redox-conjugated polynucleotide and a detection probe (e.g., a target-specific aptamer, antibody or antibody portion, and/or polynucleotide sequence that specifically binds to the target) conjugated to a polynucleotide. The redox-conjugated polynucleotide may be hybridized to the polynucleotide conjugated to the detection probe, forming the ECPA probe. The second solution may also include a buffer (e.g., HEPES buffer). The system may also include a third solution of redox-conjugated polynucleotide in buffer that does not include the detection probe.

An example of a method including the exemplary system described above may include pre-incubating the first solution (e.g., the proto nucleic acid layer) at a relatively high concentration over the electrode to form the nucleic acid layer in which the thiolated polynucleotide hybridized to the conjugated capture probe is bound to the electrode. In some examples, the second (ECPA probe) solution may be concurrently incubated with the sample to be tested, such as a blood sample, urine sample, etc., to form a solution of ECPA plus sample. This ECPA and sample solution may then be added to the pre-incubated nucleic acid layer and allowed to incubate for a predetermined time (e.g., about 5 minutes, about 10 minutes, about 15 minutes, etc.), before being washed with the third solution (of e.g., 1 nM of redox-conjugated polynucleotide, 2 nM of redox-conjugated polynucleotide, 3 nM of redox-conjugated polynucleotide, 5 nM of redox-conjugated polynucleotide, 10 nM of redox-conjugated polynucleotide, etc.) in order to stop further formation of ECPA complexes and reduce the background signal. An electrochemical signal may then be read from the electrode as described herein (e.g., at between 1-1 kHz, such as about 100 Hz) for a detection time, e.g., between 1 min-5 min, in the presence of the third solution of redox-conjugated polynucleotide. The assay may be performed at any appropriate temperature, such as, etc., room temp (e.g., 25 degrees C.), 37 degrees C., etc. (e.g., between 20-40 degrees C., between 24-40 degrees C., etc.).

The sample may comprise a biological sample selected from the group consisting of: blood serum, whole blood, nasal aspirates, saliva, urine, feces, cell lysate, dialysis sampling, tissue biopsy, cell media, and a combination thereof. In another embodiment, the biological sample is unprocessed. For example, whole blood, saliva, or urine samples that have not been processed through dilution or purification steps. In another embodiment, the method is used in a basic research laboratory to detect, quantify, or identify proteins, peptides, or cells. In another embodiment, the method is used in a clinical laboratory to detect, quantify, and/or identify biomarkers of disease. In yet another embodiment, the method is used at the point-of-care (POC) to detect, quantify, and/or identify biomarkers of disease.

Embodiments of the present disclosure include a method of detecting, identifying, and/or quantifying a single molecule of the target or a concentration of the target as low as the attomolar to millimolar range. In an embodiment, a concentration of a target in the sample as low as about 1 attomolar is detected. In an embodiment, the method is used to detect a single molecule of the target protein or peptide. In another embodiment, the method is used to detect femtomolar concentrations of the target. In another embodiment, the method is used to detect picomolar concentrations of the target. In another embodiment, the method is used to detect nanomolar concentrations of the target. In another embodiment, the method is used to detect micromolar concentrations of the target. In another embodiment, the method is used to detect millimolar concentrations of the target. Any of these methods and systems may also include detecting a target in a sample where the target is quantified using a readout method selected from surface plasmon resonance (SPR), Raman spectroscopy, and a combination thereof.

The nucleic acid layer may comprise surface immobilized DNA. The ECPA probe may comprise redox-conjugated polynucleotide and a detection probe, in which the redox-conjugated polynucleotide is a methylene blue conjugated DNA (MB-DNA). The molecular recognition elements (e.g., of the detection probe and/or the capture probe) may each independently be selected from an aptamer, an antibody, an antibody/DNA conjugate, and a combination thereof. The target may be selected from a peptide, a protein, a small molecule, a whole cell, a multi-protein complex, a nucleic acid, a virus, and a combination thereof.

For example, an electrochemical proximity assay (ECPA) may include two aptamer or antibody-oligonucleotide probes using proximity-dependent DNA hybridization to move a plurality of redox active molecule to within a predefined (and optimized) distance near a gold electrode.

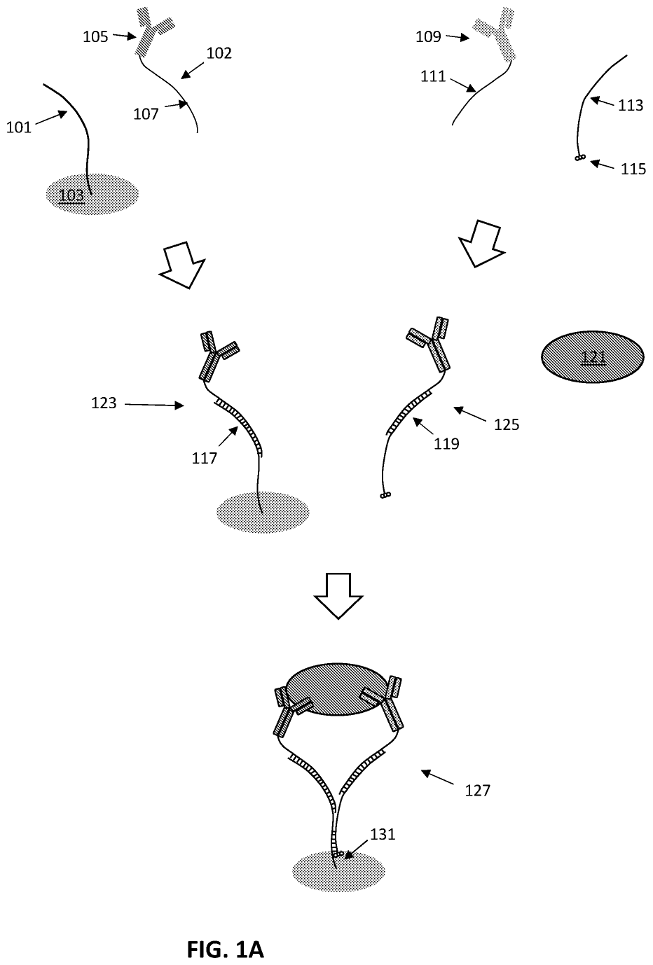

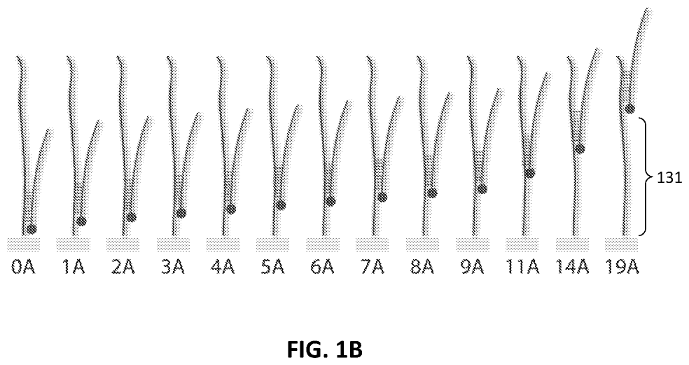

For example, FIG. 1A schematically illustrates one example of an ECPA as described herein. In FIG. 1A, the five components of the ECPA are shown: a capture probe, a capture polynucleotide (e.g., a first polynucleotide conjugated to a conductive base), a detection probe, a detection polynucleotide (e.g., a third polynucleotide), and a target. The capture probe 102, including a molecular recognition element 105 and a polynucleotide linking region 107 (e.g., a second polynucleotide), is shown being combined (e.g., hybridized) with a first polynucleotide 101 that is conjugated to an electrically conductive base 103. The polynucleotide linking region of the capture probe may be complementary to a first region 117 of the first polynucleotide, as shown in the middle region, showing the assembled nucleic acid layer 123. This crosslinking step may be performed separately and before combining with the other portions of the assay.

The detection probe 109 may be include a polynucleotide 111 having a region that is complementary to a region of the detection polynucleotide 113. One or more (e.g., 2, 3, 4, 5, 6, 7, 8, etc.) redox molecules 115 are shown coupled to the detection polynucleotide (forming a redox-conjugated polynucleotide). The detection probe may be hybridized to the redox-conjugated polynucleotide to form an ECPA probe 125, as shown. Thus, the detection probe may be coupled to the redox-conjugated polynucleotide through a region 119 of complimentary sequence.

The nucleic acid layer 123 may then be combined with the ECPA probe 125 and a target 121 to form a complex 127, as shown. When the ECPA probe is bound to the nucleic acid layer, the plurality or redox molecules are separated by spacer 131 from the conductive base by between 3 and 5 nucleotides of the polynucleotide of the nucleic acid layer, as shown. The spacer distance may be a stretch of adenosine on the first polynucleotide. Thus, in some variations the spacer distance may be optimized to be between about 1.1 nm and 1.7 nm of distance. As described in greater detail herein, the optimal spacing distance may depend upon the salt concentration of the assay and/or the optimal salt concentration may be estimated based on the spacing distance. For example, for a salt concentration of between about 0.25 to 0.75 M, the spacing between the redox molecule(s) and the substrate may be between about 3 to 5 nucleotides when generating an electrochemical signal in the conductive base. For a salt concentration of the assay between about 0.05 M to 0.25 M, the redox molecule of the ECPA probe may be optimally separated from the conductive base by greater than 7 nucleotides (e.g., 8 nucleotides, 9 nucleotides, etc.). For a high salt concertation of between about 0.75 and 1.25M, the spacing may be smaller, e.g., 4 nucleotides or less (e.g., 3 nucleotides or less, 2 nucleotides, etc.).

In general, nucleic-acid based electrochemical biosensors may use target-induced structural change in the probe for quantification. This structural change results in shift in the electrochemical signal that is proportional to the target concentration. In contrast, in ECPA, the electrochemical signal (and not the shift in the electrochemical signal) is proportional to the target concentration. Prior work on nucleic-acid based electrochemical biosensors suggested that the electrochemical response of a single redox molecule confined to an electrode surface via flexible molecular tether (e.g., using immobilized methylene blue tagged DNA) had a threshold length of about 10 times the diffusion length, and moreover, the closer to the conductive base the redox molecule was coupled through spacer 131, the better the signal. See, e.g., Huang et al. (describing the use of a random walk model). Specifically, prior to the work described herein, for ECPA it was believed that the closer the redox molecule was to the electrode surface, the higher the signal that would be produced, and that the signal would gradually decrease when placed farther from the surface.

Surprisingly, described herein are optimal distances for the redox molecule(s) that are within a specific spacing range, and in particularly where more than one (e.g., more than two, more than three, more than 4, between 2-15, between 3-15, between 4-15, 5, 6, 7, 8, etc.) nucleotide spacers are used. Distance dependence experiments on spacer 131 length were conducted, as illustrated in FIG. 1B. FIG. 1 B shows a schematic of a series of poly(A) modified thilated-DNA (e.g. polynucleotides) forming a part of a nucleic acid layer on a conductive base (e.g., gold substrate). In FIG. 1B, series of thirteen different redox-labeled polynucleotide (MB-DNA) strands having complementary regions that allow increasingly more offset binding regions (placing the redox molecule further from the surface) were examined. In this example, 10 base pair hybridization was used, and each test was hybridized overnight to suppress the interference of hybridization kinetics. Thus, the redox molecule (methylene blue) was positioned at various distances from the conductive base. The distances of the separation that are mentioned (e.g., 0A-19A, where each A corresponds to an adenine nucleotide) are units of nucleotide and represents the number of nucleotides per spacer 131. For example, in case of 0A-thiolated-DNA, the methylene blue will be closer to surface, whereas 19A thiolated-DNA places the methylene blue at gap separation distance of 19 nucleotides from the conductive surface.

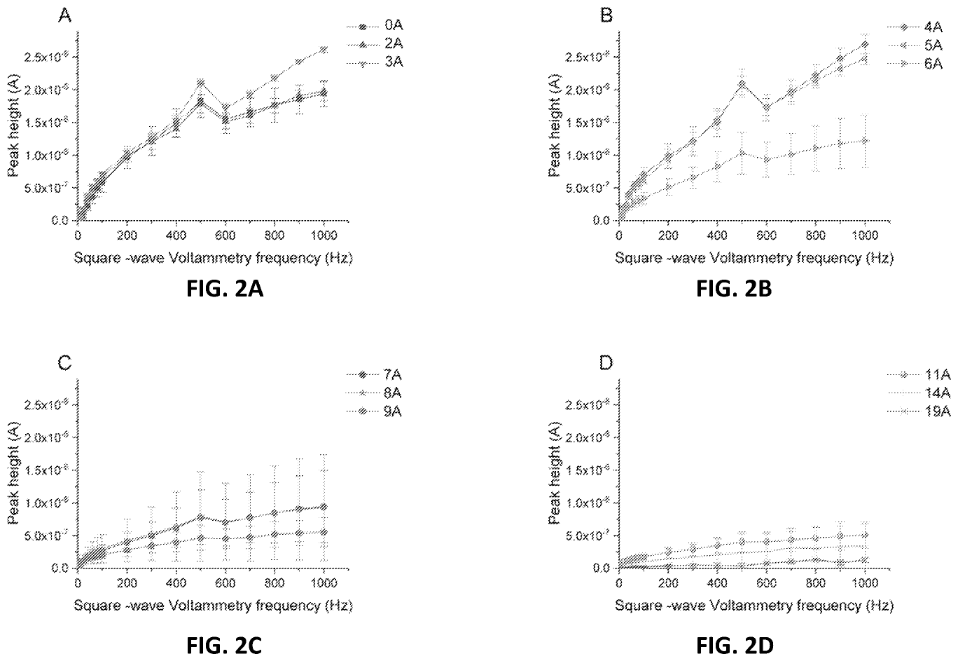

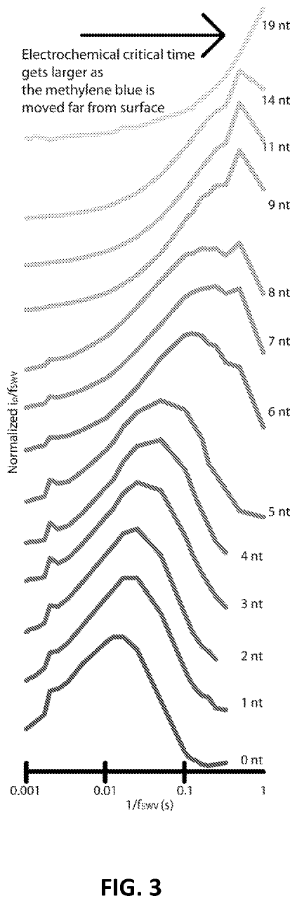

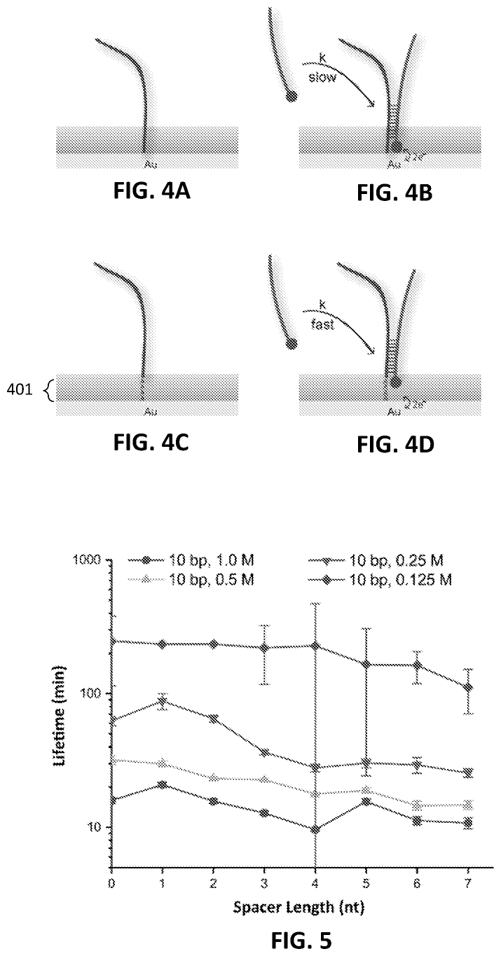

In FIG. 1B, the peak height of various distances for the redox were measured, and these results are shown in FIGS. 2A-2D. FIGS. 2A-2D show peak current versus the square wave voltammetry (SWV) frequency relationship for the different gap (spacing) distances. Peak height was measured using the frequencies between 1 and 1000 Hz. Surprisingly it was observed that the signal increased from 0A till 4A (FIGS. 2A-2B) before decreasing showing a surprisingly non-linear response with separation distance. In analyzing characteristic reaction times (FIG. 3), the value of the electrochemical critical time was increasing from 0A until 19A, with 0A showing faster kinetics than 4A. FIG. 3 shows the relationship between the peak current/frequency, and 1/f exhibits a maxima at a critical frequency related to the apparent electron transfer rate. As the separation of the redox molecule from the conductive base increased, there was a shift in this critical time from the left to the right, likely due to a decrease in the electrochemical transfer rate. Without being bound by a particular theory of operation, this initial increase in the signal may be due to a hindrance introduced by an electrical double layer on the surface towards DNA hybridization. FIGS. 4A-4D illustrates one possible model for this. In general, it was hypothesized that a double layer on the surface induces hindrance towards DNA hybridization closer to surface, resulting in a discrepancy in hybridization kinetics for similar DNA binding.

In any of the methods and systems described herein, voltammetry may be used to read and/or interpret the electrochemical signal from the ECPA sensor(s). In particular, square wave voltammetry may be used. Square wave voltammetry (SWV) is a form of linear potential sweep voltammetry that uses a combined square wave and staircase potential applied to a stationary electrode. For example, SWV may be used with a reference electrode (e.g., an Ag/AgCl reference) and a counter electrode (e.g., a platinum counter electrode). The frequency used may be, e.g. between 1 Hz and 10 kHz, such as 100 Hz. For example, current at a working electrode (including the ECPA sensor(s)) may be measured while the potential between the working electrode and a reference electrode is swept linearly in time. The potential waveform can be viewed as a superposition of a regular square wave onto an underlying staircase.

In FIGS. 4A-4D, a double layer on the surface may induce hindrance towards DNA hybridization closer to the surface. FIG. 4A shows a portion of a nucleic acid layer including a first polynucleotide conjugated to a conductive base (e.g., gold). When the ECPA probe (or partial probe), including a polynucleotide having a complementary region for hybridizing to the nucleic acid layer, is configured so that the redox molecule is close to the conductive layer (e.g., within a few nucleotides of the surface, as shown in FIG. 4B), the binding of the ECPA probe may be slow and hindered, resulting in a lower electrochemical signal, despite the close proximity, as compared to examples where the hybridization region of the nucleic acid layer is separated from the conductive surface by a distance 401, as shown in FIGS. 4C and 4D. In FIG. 4D, the hybridization may be faster, as the redox molecule and is outside of a putative double layer. Consistent with this finding, the optimal binding distance is sensitive to the salt content.

A kinetic measurement of a 10 bp hybridization region of MB-DNA with thiolated-DNA was performed at various salt and spacer lengths, as shown in the graph of FIG. 5. In FIG. 5, various average hybridization lifetimes were examined for 0A to 7A, with various sodium salt concentrations. In FIG. 5, with a 0.5 M sodium concentration, the hybridization hindrance was observed until about 4A distance. For lower concentration (e.g., 0.125 M, in which the double layer is likely to be larger) the hindrance effect was observed till 6A and for higher concentrations (e.g., 1 M, in which the double layer is likely to be thinner) no observable difference in lifetime was observed. FIG. 5 shows that the average hybridization lifetime of 10 bp DNA at various distances from the electrode with different salt concentrations altered the optimal spacing (e.g., typically with the redox molecule(s) between 3-5A, or 3-5 nucleotides from the conductive surface), when the salt concentration is between 0.1 and 1 M (e.g., between about 0.1 M and 0.8 M, between about 0.1 M and 0.75 M, between about 0.1 M and 0.7 M, between about 0.1 M and about 0.6 M, between about 0.125 M and about 0.5 M, etc.).

### Assay Optimization

In addition, the method and apparatuses described herein also teach optimization of the assay in order to reduce background. A sensitive assay may typically require the detection of target-dependent signal only in the presence of target (e.g., reducing false positives). In case of ECPA, MB-DNA hybridized to thiolated-DNA in the absence of target may generate false positives.

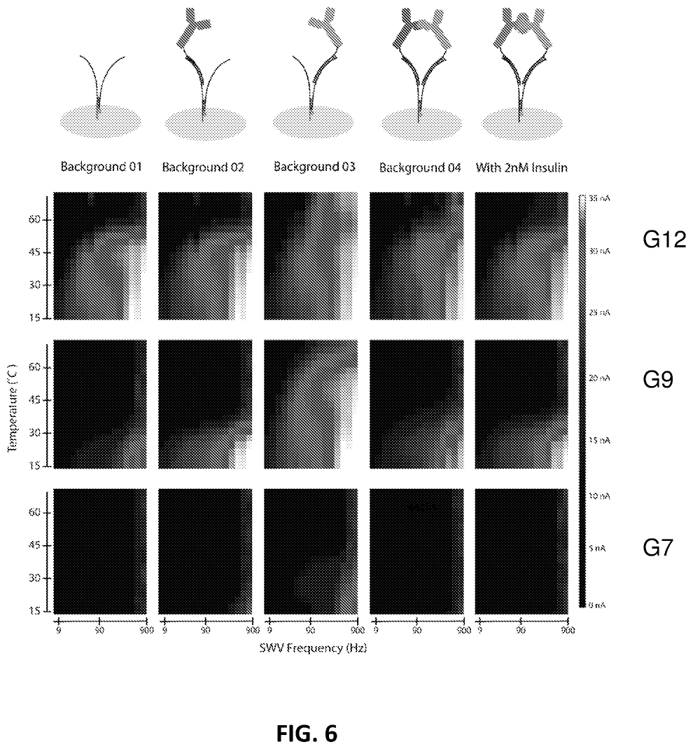

In some variations of ECPA, as described herein (see, e.g., FIG. 1), the ECPA complex may be formed by binding of five component on the conductive surface. Because of these different components, there are four possible backgrounds that may arise. FIG. 6 illustrates some of these. FIG. 6, shows an optimization experiment for one example of ECPA (e.g., insulin ECPA) with G7, G9, G12 thiolated-DNA, comparing four possible backgrounds with the signal. In G7, the nucleic acid layer hybridizes with the ECPA probe by 7 base pair. In G9, the nucleic acid layer hybridizes with ECPA probe by 9 base pair. In G12, the nucleic acid layer hybridizes with ECPA probe by 12 base pair. G7, G9 and G12 refer to the hybridization number.

In FIG. 6, the first background (Background 01) is due to hybridization of the polynucleotide coupled to the conductive base and the redox-conjugated polynucleotide. The second background (Background 02) is due to the hybridization of the nucleic acid layer with the redox-conjugate polynucleotide. The third background (Background 03) is due to the hybridization of the first polynucleotide (conjugated to the substrate) and the ECPA probe (the redox-conjugated polynucleotide and the detection probe). The fourth background (Background 04) is due to the nucleic acid layer (the capture probe and the first polynucleotide) binding to the ECPA probe (the redox-conjugated polynucleotide and the detection probe) in the absence of a target. FIG. 6 shows a heat map illustrating the amount of background current for each of these backgrounds, at a variety of different temperatures and SWV frequencies (e.g., comparing the effect of temperature, SWV Hz and different hybridization). Briefly, for an effective assay, background 01, background 02, and background 03 should be suppressed.

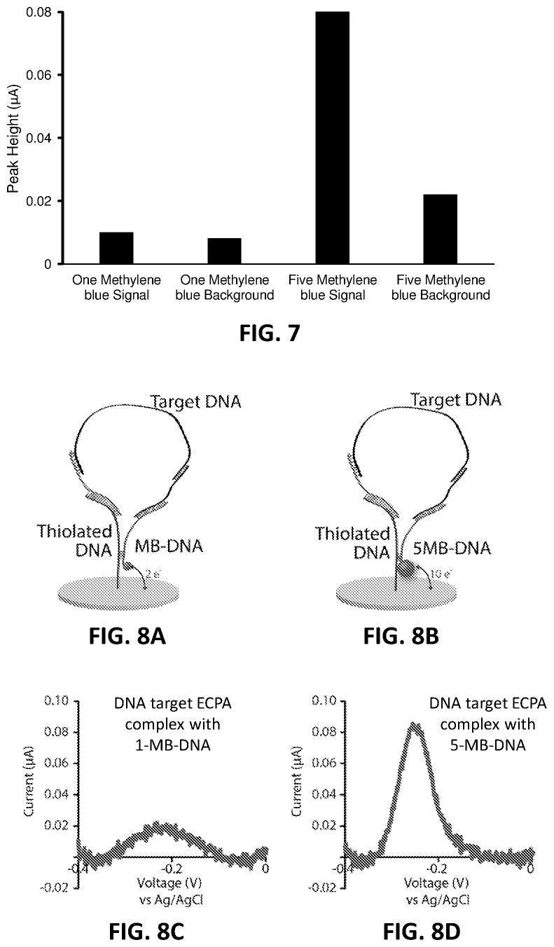

As mentioned above, multiple redox molecules may be used at the same time, which may increase the signal sensitivity. For example, FIG. 7 shows a comparison between ECPA output signals comparing a single redox molecule (e.g., one methylene blue) and multiple redox molecules (e.g., five methylene blue molecules). In FIG. 7 the model ECPA data shows both signal and background comparing one methylene blue with five methylene blue, showing a peak height for signal with five methylene blue that is eight fold higher than one methylene blue, while the background is only three fold higher. This also improves the signal to noise ratio by about three-fold.

The methods described herein may be optimized by pre-incubating the components forming the nucleic acid layer, e.g. the capture probe and the first polynucleotide, and separating pre-incubating the ECPA probe components (e.g., the redox-conjugated polynucleotide and the detection probe). The capture probe and the first polynucleotide may be incubated overnight on the surface (which may eliminate background 01 and 03) and separately (and concurrently) the ECPA probe components may be pre-hybridized (e.g., combining of AB2 with MB-DNA). This may eliminate background 01 and background 02. By following this protocol, only background 04 is left, which is comparatively weaker than other backgrounds and weaker than signal.

### Examples

As mentioned above, the ECPA methods and systems described herein may have a lower background and a higher specific signal as compared to previously described ECPA methods and systems. Specifically, the spacing the redox agent(s), and in particular, multiple redox agents from the conductive surface by 3 or more (e.g., between 3-8, between 3-5, etc.) nucleotides along the first nucleotide of the nucleic acid layer has surprisingly been found to decrease the background by more than eight-fold (e.g., between 8 times and 13 times) compared to what would be expected from previously described ECPA assays.

FIGS. 8A-8D, 9A-9D, 10, 11, 12A-12B, 13A-13B and 14A-14C described below illustrate examples of ECPA assays and systems that illustrate these advantages. These examples specifically illustrate serum amyloid A-1 (SAA-1), midregional pro-adrenomedullin (MR-proADM), and N-terminal pro-b-type natriuretic peptide (NT-proBNP) ECPA systems, however these examples are illustrative only. Other targets, including but not limited to other protein or peptide targets, and DNA targets, will benefit from the same features described herein.

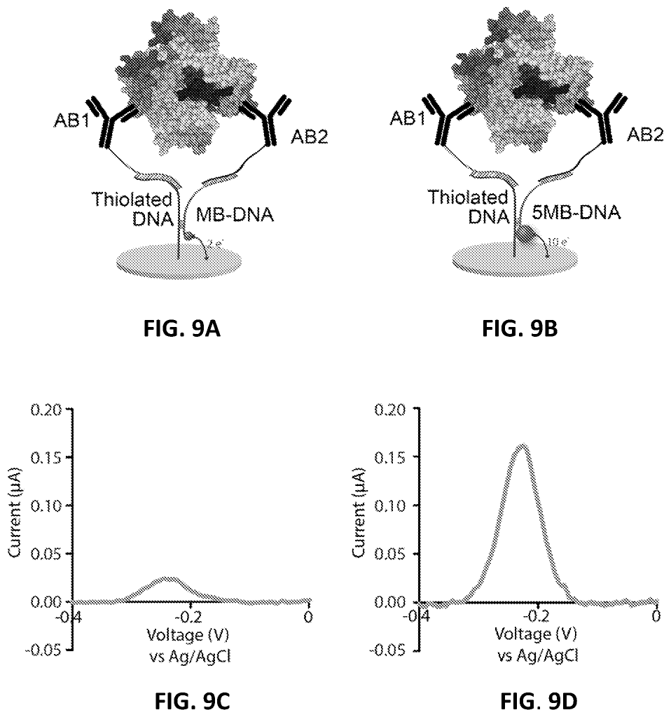

For example, FIGS. 8A-8D illustrate examples of ECPA showing a comparison between the use of a single redox molecule versus the use of five redox molecules. In this example the redox molecule is methylene blue. These methods and systems are appropriate for point of care measurements, as there is no amplification, no washing steps, and no expensive instrumentation required for sensitive measurement. In particular the improved ECPA described herein in which the redox molecule(s) are spaced, e.g., by between bout 3-5 nucleotides from the conductive base, particularly when using multiple redox molecules, may be of particular advantage. For example, these improved ECPA system may have overall larger signal output than traditional ECPA in a commercial electrode system. FIG. 8A compares the ECPA signal from a generic DNA ECPA complex, which is formed by sandwich target DNA as described above, with a single redox (1 methylene blue, shown in FIGS. 8A) and multiple redox molecules (5 methylene blue, shown in FIG. 8B). The signal from the multivalent in FIG. 8B complex is approximately 13-fold higher than the single redox molecule. A similar response is observed with a serum amyloid A-1 ECPA sensor, shown as illustrated in FIGS. 9A-9D. In FIGS. 8C and 8D, the graphs shown in square-wave voltammetry (SWV) measurements with Ag/AgCl as reference and platinum as counter electrodes (100 Hz is SWV frequency) for single redox molecule (1 MB) and multiple redox molecule (e.g., 5 MB) as discussed above.

Similar results were shown for the SAA-1 ECPA sensors shown in FIGS. 9A and 9B. In FIG. 9A the SAA-1 ECPA sensor includes a single methylene blue, spaced 4 nucleotides (4A) from the conductive base, while in FIG. 9B the SAA-1 ECPA sensor is identical, but includes five methylene blue (5 MB), also spaced 4 nucleotides (4A) from the conductive base. As shown in FIGS. 9C-9D, the sensed signal is more than five times larger in the 5 MB sensor.

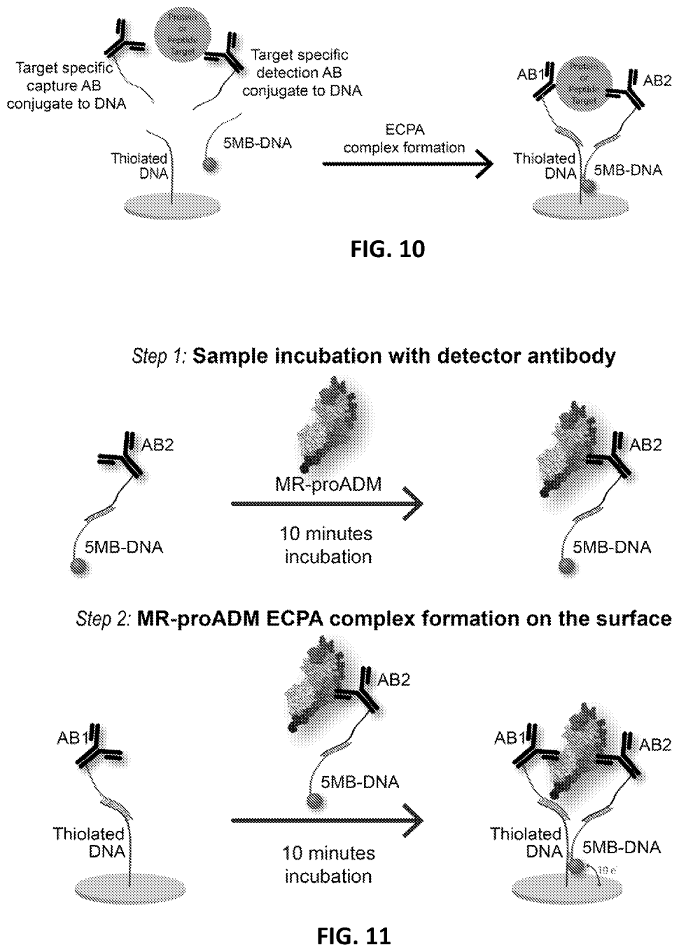

As mentioned, these features may be generically applied to ECPA sensors. FIG. 10 shows a general case of the assay method and assay system. Any target for which two probes (e.g., antibodies, aptamers, or a combination of both) that binds to the target in non-overlapping regions may be quantified by the ECPA methods and systems described herein. In FIG. 10, a model for constructing the ECPA complex for a new target is illustrated; in the case of antibodies, the capture antibodies and detection antibodies are conjugated with DNA which can hybridize with thiolated-DNA and signaling DNA (e.g., 5 MB-DNA), respectively.

In another example similar to that shown in FIG. 10, a first solution includes a thiolated first polynucleotide that is hybridized to a capture probe to form the proto nucleic acid layer. This is illustrated in FIG. 11. Although FIG. 10 shows preincubation of the thiolated first polynucleotide (e.g., thiolated-DNA) with the electrode before it hybridizes to the capture probe, in some examples the thiolated first polynucleotide is hybridized to the capture probe to form a proto nucleic acid layer before adding to the electrode. This proto nucleic acid layer is then applied (typically at a relatively high concentration, such as about 0.1 mM or greater, 1 mM or greater, 5 mM or greater, 10 mM or greater, 20 mM or greater, 100 mM or greater, etc.) to the electrode and incubated (“preincubated”) to form the nucleic acid layer in which the thiolated first polynucleotide that is hybridized to the capture probe is bound to the electrode.

Concurrently with incubation to form the nucleic acid layer, the sample to be tested may be combined with a second solution including the ECPA probe and incubated (“preincubated”). The ECPA probe may include the detection probe conjugated to a fourth polynucleotide and hybridized to a redox conjugated third polynucleotide that may include multiple redox molecules (e.g., multiple methylene blue molecules, such as 5 methylene blue). Although the left side of FIG. 10 shows an example in which the redox conjugated third polynucleotide is not yet hybridized to the fourth polynucleotide coupled to the detection probe, in some examples the second solution includes assembled ECPA probe before combining with the sample to be tested for a target (e.g., protein or peptide target, polynucleotide target, etc.). The sample (e.g., blood, urine, etc.) may be combined with the second (ECPA probe in buffer) solution at the same time that the first solution is added to the electrode to form the nucleic acid layer. The pre-incubation time may be, e.g., between 1-90 minutes, between about 5-30 minutes, between about 10-20 minutes, etc.

The ECPA and sample solution may then be added to the nucleic acid layer and allowed to incubate for a predetermined time (e.g., about 5 minutes, about 10 minutes, about 15 minutes, etc. such as between 1-60 minutes, between 2-30 minutes, between 3-20 minutes, etc.), before being washed with a third solution of the redox-conjugated polynucleotide (e.g., about 1-10 nM redox-conjugated polynucleotide in HEPES buffer) to stop further formation of ECPA complexes and reduce the background signal. An electrochemical signal may then be read from the electrode as described herein (e.g., at between 1-1 kHz, such as about 100 Hz) for a detection time, e.g., between 1 min-5 min, in the presence of the third solution of redox-conjugated polynucleotide.

FIG. 11 schematically illustrates an ECPA method as described. In this example, the electrodes are initially preincubated with a capture antibody (proto nucleic acid layer solution) as described above, so that the thiolated-DNA is immobilized on the surface of a gold electrode where the primary (capture) antibody (AB1) that is conjugated to a polynucleotide is already hybridized to the thiolated DNA. The sample solution is prepared by mixing the signaling polynucleotide (e.g., 5 MB-DNA) with oligomer-conjugated detection antibody. These two hybridize to form a single unit (ECPA probe).

As shown in FIG. 11, the ECPA method is a two-step process. A sample (e.g., 5 μL) may be mixed with the ECPA probe mixture (in some examples in equal parts, e.g., 5 μL) and may be incubated for 10 minutes, so that the target is bound to the detection probe (shown here as a detection antibody). Next, this sample reagent mixture may be introduced to the nucleic acid layer, including the electrode and the capture probe, and incubated, e.g., for about 10 minutes. In this incubation, the capture antibody may sandwich the target with the detection antibody, initiating allosteric hybridization of the thiolated-DNA with 5 MB-DNA, so that the redox molecules are closer to the surface. The current peak height from the square-wave voltammetry measurement may be proportional to the target concentration. FIG. 11 illustrates specifically an MR-proADM example. The same technique may be applied to any other ECPA target. The incubation time(s) may be optimized for other targets.

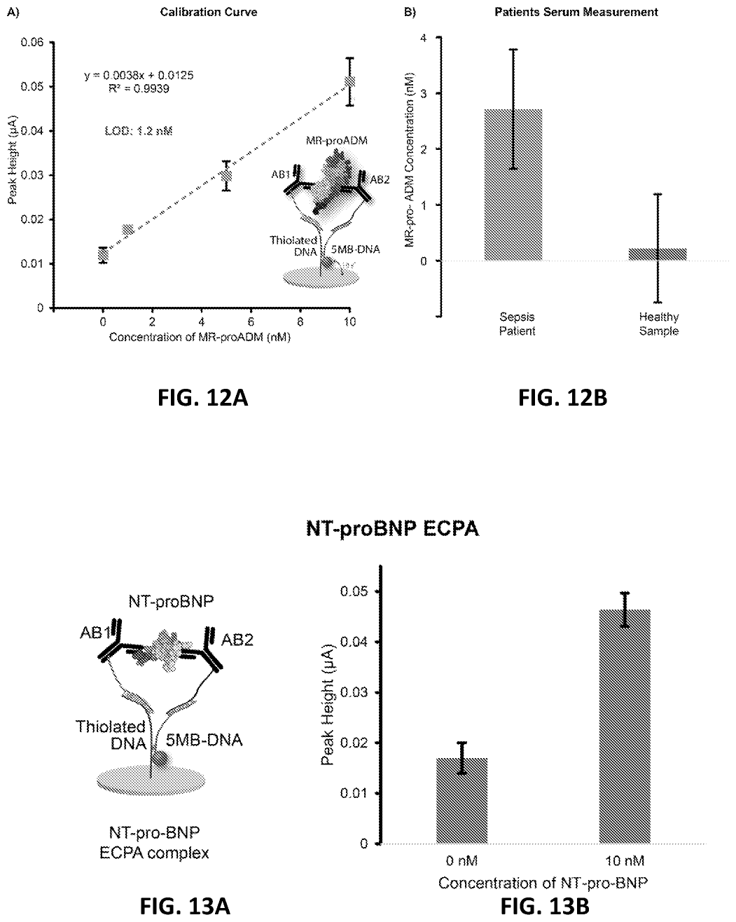

This two-step protocol was used to quantify the MR-pro-ADM, and the results are shown in FIGS. 12A-12B. FIG. 12A shows a calibration curve of midregional pro-adrenomedullin (MR-proADM) in a buffer. A limit-of-detection (LOD) of 1.2 nM was achieved. To test the suitability of the assay at a point of care (PoC), an undiluted serum from sepsis patients (Discovery Life Sciences) was tested. As shown in FIG. 12B, a comparison between sepsis patients (n=4) and healthy samples (n=4), showed that sepsis patients had a significantly elevated level of MR-pro-ADM. The MR-pro-ADM ECPA system in this example also included five redox molecules (5 MB) spaced by 4A from the conductive base.

Another example assay is shown in FIGS. 13A-13B for NT-proBNP. In this example, the N-terminal pro-b-type natriuretic peptide (NT-proBNP) ECPA model (shown in FIG. 13A) also had five methylene blue molecules spaced by a 4 A (four nucleotides) from the conductive base. As shown in FIG. 13B, a preliminary measurement of NT-proBNP ECPA comparing no NT-proBNP (0 nM) and 10 nM NT-proBNP SWV peak height, showed that the higher concentration of the samples had a significantly higher signal than the absence of the target. In FIG. 13B 3 examples of each were tested.

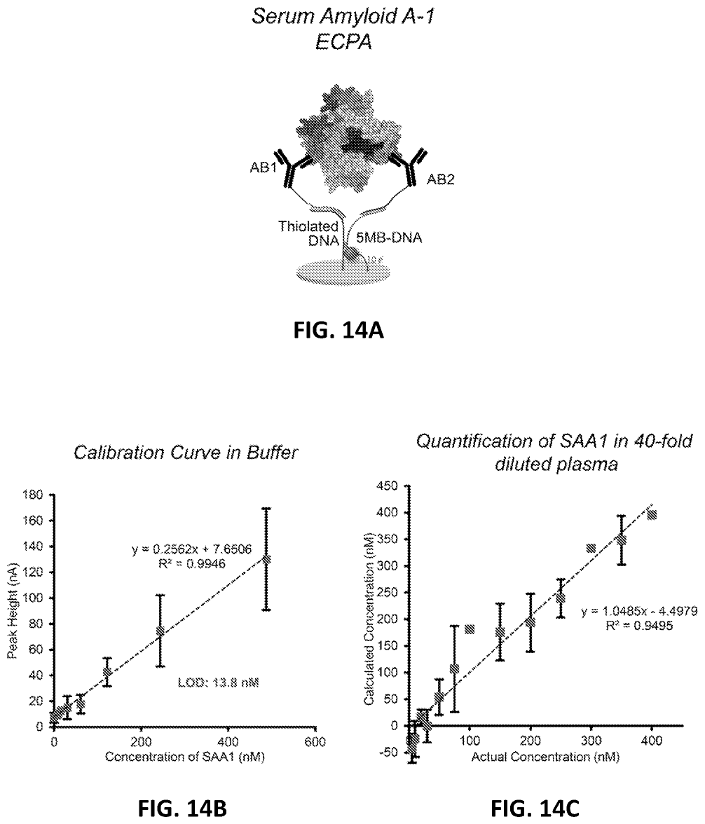

FIGS. 14A-14C illustrate results of tests performed on an SAA-1 ECPA system. In this example Serum amyloid A-1 (SAA-1) protein was quantified using ECPA with 5 MB-signalling DNA, also spaced by 4A, shown schematically in FIG. 14A. The two-step protocol described above was used to quantify the SAA-1. FIG. 14B shows the calibration curve in the buffer, in which SWV measurement with 100 Hz, resulted in a LOD of 13.8 nM. The suitability of the assay in point-of-care measurement was assayed by testing spiked samples, and these results are shown in FIG. 14C. In this example, SAA-1 was spiked in 40-fold diluted plasma (R2 diagnostics) and tested. FIG. 14C shows the comparison between calculated concentration (calculated from the calibration curve of FIG. 14B) and actual concentration in plasma (as spiked). As shown good agreement was found over the entire scope of the range tested, representing a biologically relevant range.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An electrochemical proximity assay (ECPA) method comprising:
exposing a mixture of an ECPA probe and a target to a conductive base onto which a nucleic acid layer has been formed, wherein the ECPA probe comprises a polynucleotide coupled to a redox molecule;
generating an electrochemical signal in the conductive base by forming a complex of the nucleic acid layer, the ECPA probe, and the target and binding the polynucleotide of the ECPA probe to a complementary polynucleotide of the nucleic acid layer on the conductive base, so that the redox molecule of the ECPA probe is separated from the conductive base by between 3-8 nucleotides of the nucleic acid layer; and
quantifying an amount of the target by analyzing the electrochemical signal, wherein the electrochemical signal changes in proportion to changes in concentration of the target.

2. The method of claim 1, wherein the redox molecule is methylene blue.

3. The method of claim 1, wherein the redox molecule of the ECPA probe is separated from the conductive base by between 3 to 5 nucleotides of the nucleic acid layer when generating an electrochemical signal in the conductive base and a salt concentration is between 0.25 to 0.75 M.

4. The method of claim 1, wherein the redox molecule of the ECPA probe is separated from the conductive base by between 5 to 8 nucleotides of the nucleic acid layer when generating an electrochemical signal in the conductive base and a salt concentration is between 0.05 to 0.25 M.

5. The method of claim 1, wherein the nucleic acid layer comprises a thiolated first polynucleotide hybridized to a target-specific capture probe.

6. The method of claim 1, wherein the ECPA probe comprises the polynucleotide coupled to a plurality of redox molecules at a 3′ end of the polynucleotide.

7. The method of claim 1, wherein the ECPA probe comprises between 1-14 additional redox molecules, wherein the additional redox molecule are separated from the conductive base by between 3-5 nucleotides when the ECPA probe is bound to the nucleic acid layer.

8. An electrochemical proximity assay (ECPA) method comprising:
pre-incubating a capture probe and a thiolated first polynucleotide with a conductive base to form a nucleic acid layer, wherein the capture probe comprises a molecular recognition element that specifically binds to a target and coupled to a second polynucleotide having a first region that is complementary to a second region of the first polynucleotide;
exposing a mixture of an ECPA probe and the target to the nucleic acid layer, wherein the ECPA probe comprises a third polynucleotide coupled to a plurality of redox molecules at a 3′ end of the third polynucleotide;
generating an electrochemical signal in the conductive base by forming a complex of the nucleic acid layer, the ECPA probe, and the target and binding the third polynucleotide of the ECPA probe to a complementary polynucleotide of the nucleic acid layer, so that the redox molecules of the ECPA probe are separated from the conductive base by between by 3 to 8 nucleotides of the first polynucleotide; and
quantifying an amount of the target by analyzing the electrochemical signal, wherein the electrochemical signal changes in proportion to changes in concentration of the target.

9. An electrochemical proximity assay (ECPA) method comprising:
pre-incubating a capture probe and a thiolated first polynucleotide with a conductive base to form a nucleic acid layer, wherein the capture probe comprises a molecular recognition element that specifically binds to a target and coupled to a second polynucleotide having a first region that is complementary to a second region of the first polynucleotide;
separately pre-incubating a redox-conjugated polynucleotide and a detection probe to form an ECPA probe, wherein the redox-conjugated polynucleotide comprises one or more redox molecules conjugated to a third polynucleotide and wherein the detection probe comprises a molecular recognition element that specifically binds to the target and coupled to a fourth polynucleotide having a third region that is complementary to a fourth region of the redox-conjugated polynucleotide; and
combining the ECPA probe and the nucleic acid layer;
generating an electrochemical signal in the conductive base by forming a complex of the nucleic acid layer, the ECPA probe, and the target and binding the redox-conjugated polynucleotide of the ECPA probe to a fifth region of the first polynucleotide, so that the one or more redox molecules of the ECPA probe are separated from the conductive base by between 3 to 8 nucleotides of the first polynucleotide of the nucleic acid layer; and
quantifying an amount of the target by analyzing the electrochemical signal, wherein the electrochemical signal changes in proportion to changes in concentration of the target.

10. The method of claim 9, wherein the one or more redox molecules is methylene blue.

11. The method of claim 9, wherein one or more redox molecules of the ECPA probe is separated from the conductive base by between 3 to 5 nucleotides of the first polynucleotide of the nucleic acid layer when generating an electrochemical signal in the conductive base and a salt concentration is between 0.25 to 0.75 M.

12. The method of claim 9, wherein the one or more redox molecules of the ECPA probe is separated from the conductive base by between 5 to 8 nucleotides of the first polynucleotide of the nucleic acid layer when generating an electrochemical signal in the conductive base and a salt concentration is between 0.05 to 0.25 M.

13. The method of claim 9, further comprising rinsing the complex of the nucleic acid layer, the ECPA probe, and the target with a solution of the redox-conjugated polynucleotide before quantifying the amount of the target.

14. The method of claim 9, wherein the ECPA probe comprises between 3 and 15 redox molecules that are separated from the conductive base by between 3 to 5 nucleotides when the ECPA probe is bound to the nucleic acid layer.

15. The method of claim 9, wherein the molecular recognition element of the capture probe comprises one or more of: an aptamer, an antibody, and an a single-stranded polynucleotide.

16. The method of claim 9, wherein pre-incubating the capture probe comprises pre-incubating for less than 2 hours.

17. An electrochemical proximity assay (ECPA) system comprising:
a nucleic acid layer comprising a capture probe and a first polynucleotide conjugated to a conductive base, wherein the capture probe comprises:
a molecular recognition element configured to specifically bind to a target, and
a second polynucleotide having a first region that is complementary to a second region of the first polynucleotide; and
an ECPA probe comprising a redox-conjugated polynucleotide conjugated to a detection probe, wherein the redox-conjugated polynucleotide comprises a plurality of redox molecules conjugated to a third polynucleotide, and wherein the detection probe comprises a molecular recognition element that specifically binds to the target and coupled to a fourth polynucleotide having a third region that is complementary to a fourth region of the redox-conjugated polynucleotide,
wherein the ECPA probe and the nucleic acid layer are configured to form a complex with the target wherein the redox molecule of the ECPA probe is separated from the conductive base by between 3 to 8 nucleotides of the first polynucleotide of the nucleic acid layer.

18. The system of claim 17, wherein the plurality of redox molecules comprises methylene blue.

19. The system of claim 17, wherein the plurality of redox molecules of the ECPA probe are separated from the conductive base by between 3 to 5 nucleotides of the first polynucleotide of the nucleic acid layer when the ECPA probe and the nucleic acid layer forms a complex with the target.

20. The system of claim 17, further comprising a buffer solution having a salt concentration of between 0.25 to 0.75 M.

21. The system of claim 20, wherein the plurality of redox molecules of the ECPA probe are separated from the conductive base by between 5 to 8 nucleotides of the nucleic acid layer when generating an electrochemical signal in the conductive base and a salt concentration is between 0.05 to 0.25 M.

22. The system of claim 17, wherein the nucleic acid layer comprises thiolated-DNA.

23. The system of claim 17, wherein the ECPA probe comprises between 3 and 15 redox molecules.

24. The system of claim 17, wherein the molecular recognition element of the capture probe comprises one or more of: an aptamer, an antibody, and an a single-stranded polynucleotide.

25. An electrochemical proximity assay (ECPA) system comprising:
an assay chamber comprising a nucleic acid layer comprising a capture probe and a first polynucleotide conjugated to a conductive base of the assay chamber, wherein the capture probe comprises:
a molecular recognition element configured to specifically bind to a target, and
a second polynucleotide having a first region that is complementary to a second region of the first polynucleotide; and
a first solution comprising an ECPA probe comprising a redox-conjugated polynucleotide conjugated to a detection probe, wherein the redox-conjugated polynucleotide comprises a plurality of redox molecules conjugated to a third polynucleotide, and wherein the detection probe comprises a molecular recognition element that specifically binds to the target and coupled to a fourth polynucleotide having a third region that is complementary to a fourth region of the redox-conjugated polynucleotide,
wherein the ECPA probe and the nucleic acid layer are configured to form a complex with the target wherein the plurality of redox molecules of the ECPA probe are separated from the conductive base by between 3 to 5 nucleotides of the first polynucleotide of the nucleic acid layer.
